(19) United States
(12) Patent Application Publication
(10) Pub. No.: US 2022/0120735 A1
(43) Pub. Date: Apr. 21, 2022

(54) Testing Methods for Determination of T2R Phenotype and Applications Thereof

(71) Applicant: Phenomune, LLC, The Woodlands, TX (US)

(72) Inventor: Henry P. Barham, Baton Rouge, LA (US)

(21) Appl. No.: 17/500,755

(22) Filed: Oct. 13, 2021

Related U.S. Application Data
(60) Provisional application No. 63/092,279, filed on Oct. 15, 2020.

(51) Int. Cl. G01N 33/50; G01N 33/68

(52) U.S. Cl. CPC G01N 33/5041; G01N 2333/705; G01N 33/6842

(57) ABSTRACT

This invention provides test methods and test kits for determination of T2R phenotype.

### REFERENCE TO RELATED APPLICATION

This application claims priority from U.S. Application No. 63/092,279, filed Oct. 15, 2020, the disclosure of which is incorporated herein by reference.

### TECHNICAL FIELD

This invention relates to the stimulation of T2Rs and SCCs and applications of stimulations of T2Rs and SCCs.

### BACKGROUND

In humans, bitter and sweet taste perception is governed by G-protein coupled receptors originally identified in oral taste bud type II cells. Receptors belonging to Taste Receptor Family-1 subtypes 2 and 3 (T1R2/T1R3) detect sweet compounds such as glucose and sucrose. Greater than 50 Taste Receptor Family-2 (T2Rs) have been characterized. Stimulation of T2Rs activates the canonical taste signaling cascade involving phospholipase C β2 (PLCβ2) and transient receptor potential cation channel subfamily M member 5.

T2Rs are genetically diverse, a phenomenon that helps to explain the wide variety of taste preference both within and between cultures. Many individuals find various bitter foods to be detestable, while others do not have the same aversive response. This genetic variation of T2Rs is found on the tongue, but not exclusively. More recently, bitter and sweet receptors have been discovered in a variety of extra-oral tissues including the brain, thyroid, pancreas, testes and throughout the respiratory and gastrointestinal tracts.

A growing body of literature has suggested a role for bitter taste receptors (T2Rs) and solitary chemosensory cells (SCCs) in sinonasal innate immunity, and that genetically heritable differences in bitter taste receptors contribute to the pathogenesis of rhinosinusitis and upper respiratory tract infections. Extraoral bitter taste receptors present on ciliated mucosal cells and SCCs are known to play a role in innate immune system activity. T2R receptors are also present in the airway and appear to play a key role in respiratory defense. Various reagents, compounds, and chemicals agonize T2Rs, causing the release of products which help to clear and kill pathogens in the human respiratory mucosa.

A need therefore continues to exist for a simple test method that can provide both subjective and objective responses to evaluate levels of phenotypic expression of T2Rs and SCCs, to help predict susceptibility and clinical course of microbial infections in humans.

### SUMMARY OF THE INVENTION

This invention provides test methods to determine T2R phenotype, methods for evaluating expression or functionality of T2Rs and/or SCCs, test kits to perform T2R and/or SCC phenotype testing, and methods that may improve T2R and/or SCC phenotype for patients having certain phenotypes. Stimulation of T2Rs and/or SCCs with one or more agonists will result in an increased level of expression, leading to improved function, improving innate immune fitness to upper respiratory infections, including SARS-CoV-2, influenza, and to other viral and bacterial agents.

An embodiment of this invention is a test method for detecting byproducts released by the T2Rs and/or SCCs as a result of stimulation.

Another embodiment of this invention is a test method for determining a level of phenotypic expression of T2Rs and/or SCCs in a human subject.

Still another embodiment of this invention is a method for evaluating a level of expression or functionality of T2Rs and/or SCCs in a human subject.

Yet another embodiment of this invention is a test kit for determining T2R phenotype.

These and other embodiments and features of this invention will be still further apparent from the ensuing description and appended claims.

### FURTHER DETAILED DESCRIPTION OF THE INVENTION

As used throughout this document, the term “microbial infections” refers to both infections caused by bacteria and by viruses, unless otherwise specified.

In some art areas, the word “byproduct” refers to ancillary products of a reaction that are not of interest; however, as used throughout this document, the word “byproduct” refers to ancillary products that are of interest.

Throughout this document, the terms “interacting” and “interaction” in reference to the reagent and the byproducts released by the T2Rs and/or SCCs as a result of their stimulation refers to association, contact, and/or reaction, as applicable, as long as the interaction results in a detectable phenomenon.

Most of the methods of this invention comprise stimulating T2Rs and/or SCCs of a human subject with one or more agonists. In test methods for determining a level of phenotypic expression of T2Rs and/or SCCs in a human subject, the stimulating involves exposing at least a portion of oral or nasal tissue of the human subject to the one or more agonists.

Some of the methods of the invention also comprise detecting one or more byproducts released by the T2Rs and/or SCCs as a result of stimulation of the T2Rs and/or SCCs. One detecting method comprises i) applying one or more reagents which interact with one or more of the byproducts released by the stimulation of the T2Rs and/or SCCs, and ii) detecting the interaction of the reagent with one or more byproducts, optionally by employing chemiluminescence, an electrochemical sensor, or an optical sensor, to detect the interaction of one or more reagents with one or more of the byproducts. Another method comprises employing chemiluminescence, an electrochemical sensor, or an optical sensor to detect one or more of the byproducts released by the stimulation of the T2Rs and/or SCCs.

Detection of the interaction of the reagent with one or more byproducts released by the T2Rs and/or SCCs as a result of their stimulation comprises visual detection, taste, or both.

One of the test methods of this invention comprises stimulating T2Rs and/or SCCs of a human subject with one or more agonists, and detecting one or more byproducts released by the T2Rs and/or SCCs as a result of stimulation of the T2Rs and/or SCCs by one of the detecting methods described above.

In some methods embodying an aspect of the invention, the agonists are separately applied prior to applying the reagent which interacts with one or more byproducts released by T2Rs and SCCs as a result of stimulation of T2Rs and/or SCCs. In this test method, the reagent in one embodiment is contained in or on a test medium. In other methods embodying an aspect of this invention, the agonists and the reagents which interact with one or more of the byproducts released by T2Rs and SCCs as a result of stimulation of T2Rs and/or SCCs are contained in or on one or more test media. In other embodiments according to one aspect of the invention, an agonist and a reagent are present on or in the same test medium.

In another method of this invention, for evaluating a level of expression or functionality of T2Rs and/or SCCs in a human subject, the method comprises stimulating T2Rs and/or SCCs of a human subject with one or more agonists, and detecting one or more byproducts released by the T2Rs and/or SCCs as a result of stimulation of the T2Rs and/or SCCs. One detecting method comprises i) applying one or more reagents which interact with one or more of the byproducts released by the stimulation of the T2Rs and/or SCCs, and ii) detecting the interaction of the reagent with one or more byproducts, optionally via chemiluminescence, an electrochemical sensor, or an optical sensor. Another method comprises employing chemiluminescence, an electrochemical sensor, or an optical sensor to detect one or more of the byproducts released by the stimulation of the T2Rs and/or SCCs. An additional step in one particular aspect of this method is discerning a level of byproducts produced by the stimulation of the T2Rs and/or SCCs that is indicative of level of expression or functionality of the T2Rs and/or SCCs, which level of expression or functionality of the T2Rs and/or SCCs is correlated to a level of phenotypic expression.

In test methods of this invention for determining a level of phenotypic expression, the stimulating of T2Rs and/or SCCs of a human subject comprises exposing at least a portion of oral or nasal tissue of the human subject to one or more agonists. This method also includes recording a discerned level of taste perception by the human subject after the stimulation, and correlating the discerned level of taste perception by the human subject to a level of phenotypic expression of the T2Rs and/or SCCs of the human subject.

In some embodiments, the test method for determining a level of phenotypic expression is repeated two or more times, and in other embodiments this test method is repeated one or more times using a therapeutic agonist. In another embodiment in which the test method for determining a level of phenotypic expression is repeated one or more times using a therapeutic agonist, the repeating of the test method can be used to obtain a data set. Optionally, trend analysis is performed on the data set.

When repeating the test method for determining a level of phenotypic expression is performed two or more times, the repeating is preferably at regular time intervals. The time intervals each can be 8 hours, daily, weekly, biweekly, monthly, bimonthly, semiannually, annually, or biannually.

In a preferred embodiment of the test method for determining a level of phenotypic expression, the stimulating by each of one or more different agonists is sequential, the recording of each discerned level of taste perception by the human subject occurs after each stimulation, and the correlating is of one or more of the discerned levels of taste perception to a level of phenotypic expression of the T2Rs and/or SCCs of the human subject. In other preferred embodiments, the correlating comprises employing a computer processor programmed with machine-readable instructions that cause the computer processor to a) receive and store the discerned levels of taste perception with respect to each agonist, b) ascribe a weighting to each of the agonists according to their known stimulation of T2Rs and/or SCCs, and c) calculate a weighted taste perception from the discerned level of taste perception by multiplying the ascribed weighting and discerned level of taste perception for each agonist applied, to produce an aggregated, weighted level of taste perception which indicates the level of phenotypic expression.

In the practice of this invention when a reagent is applied, an agonist may be separately applied prior to applying the reagent. When the byproduct with which the reagent interacts decays relatively quickly (e.g., nitric oxide), the reagent is best applied during or soon (e.g., less than a minute) after the agonist is applied. In these embodiments, the test medium does not contain one or more agonists but does contain one or more reagents which interact with one or more of the byproducts released by T2Rs and SCCs as a result of stimulation. In some embodiments, a test medium containing the reagent that interacts with one or more byproducts can be applied without use of an agonist, for example, when a patient has an infection known to stimulate T2Rs and SCCs and release the byproduct(s) with which the reagent will interact; however, it is recommended that an agonist be used to ensure a measurable response.

In some methods of this invention, especially when determining levels of phenotypic expression, the use of two or more different agonists is preferred because the accuracy of the determination of the level of phenotypic expression is higher when more than one agonist is employed to stimulate the T2Rs and/or SCCs. Use of one agonist provides levels of phenotypic expression results with an accuracy of about 50% to about 60%; use of two different agonists provides levels of phenotypic expression results with an accuracy of about 70% to about 80%; use of three different agonists has been found to provide level of phenotypic expression results with an accuracy of about 94% or more. Using four or more agonists does not significantly improve accuracy. Use of two or three different agonists is preferred; use of three different agonists is more preferred. Normally and preferably, a control test run using a blank that does not contain an agonist is also performed.

Stimulation of T2Rs and/or SCCs of a human subject by one or more agonists can occur via topical, nasal, oral, gastrointestinal, and/or systemic application of the agonists. In some embodiments, the stimulation comprises exposing the breath, saliva or respiratory mucosa to one or more agonists. Suitable test media may vary with the nature of the agonist or reagent present therein or thereon as well as the locus of application. When the agonist is orally applied, typical test media include paper strips, tongue depressors, and cotton swabs. In some aspects of the invention, paper strips are used as the test medium.

Several embodiments of the test methods of this invention comprise using agonists to stimulate bitter taste receptor(s) (T2Rs, especially TAS2Rs, and/or SCCs). Large numbers of agonists for T2Rs and SCCs are known. Common agonists for stimulation of T2Rs and/or SCCs include, for example, caffeine, denatonium (salts), strychnine, quinine, phenylthiocarbamate (PTC), and thiourea. Two or more agonists can be used. In preferred embodiments of the invention, the agonist is perceivable as taste by a human being via stimulation of T2Rs and/or SCCs. Agonists for therapeutic use are not necessarily the same as agonists used to stimulate the T2Rs and SCCs for testing purposes, although some agonists are suitable for both testing purposes and therapeutic use. Therapeutic agonists for stimulation of T2Rs and/or SCCs include vitamins, nitric oxide releasers, caffeine, denatonium, strychnine, quinine, xylitol, grapefruit seed extract or naringenin, or a terpene. Other therapeutic agonists are found in edible foods such as broccoli. Two or more agonists can be used when a therapeutic agonist is used. While many other agonists for T2Rs an SCCs are known, the agonists listed here are inexpensive and readily available.

Typically, when stimulated, T2Rs and SCCs produce either antimicrobial peptides or nitric oxide. The reagent which interacts with one or more byproducts released by T2Rs and SCCs as a result of stimulation interacts with the antimicrobial peptides or nitric oxide, or another byproduct. Often the reagent comprises a Griess reagent. The reagent interaction with one or more byproducts can indicate functionality of the receptors (whether or not a byproduct is released); or reagent interaction with one or more byproducts can indicate a qualitative or quantitative level of response.

In some method embodiments of this invention, pre-testing with the reagent that interacts with one or more byproducts of the stimulation of T2Rs and SCCs before applying an agonist to determine a baseline is useful.

The phrase “one or more reagents which interact with one or more of the byproducts released by T2Rs and SCCs as a result of stimulation” includes combinations of reagents when more than one reagent is needed to provide a detectable phenomenon. In some embodiments, the detectable phenomenon is for example a detectable color change. The color change can be determinable by spectroscopy or visually detectable (visible to human eyes of normal visual acuity).

In the practice of this invention, a discerned level of taste perception by the human subject after stimulation of the T2Rs and/or SCCs by an agonist refers to the intensity or strength of the taste, or an absence of taste, as perceived by the human subject. The discerned level of taste perception as perceived by the human subject is correlated to a level of phenotypic expression based on the agonist used to stimulate the T2Rs and/or SCCs, and known phenotype expressions for the discerned level of taste perception reported by the human subject.

For the discerning of a level of byproducts produced by stimulation of the T2Rs and/or SCCs, indicative of level of expression or functionality of the T2Rs and/or SCCs, which level of expression or functionality of the T2Rs and/or SCCs is correlated to the level of phenotypic expression, the discerned level of byproducts produced is correlated to the level of phenotypic expression based on the agonist used to stimulate the T2Rs and/or SCCs, and known levels of expression or functionality of the T2Rs and/or SCCs for the discerned level of byproducts produced, an in turn the known phenotype expressions for the discerned levels of expression or functionality of the T2Rs and/or SCCs.

In another embodiment of the invention, a test kit is provided. The test kit comprises at least one test medium containing (a) one or more agonists for T2Rs and/or SCCs of a human subject and/or (b) one or more reagents which interact with one or more of the byproducts released by T2Rs and SCCs in a human subject as a result of stimulation of the T2Rs and/or SCCs.

In some embodiments of the test kits of the invention, the present invention provides at least one test medium containing or coated in one or more agonists for T2Rs and/or SCCs of a human subject. In some embodiments, the test medium comprises (a) one or more agonists for T2Rs and/or SCCs, which agonist is perceivable by a human subject as taste (a subjective criterion) via stimulation of the receptors, and/or (b) one or more reagents which interact with one or more of the byproducts released by the T2Rs and SCCs of a human subject as a result of stimulation of the T2Rs and/or SCCs (an objective criterion). The interaction of the reagent with one or more byproducts of the stimulation of the T2Rs and/or SCCs provides a detectable phenomenon that is an objective criterion, for example, a color change, when interacting with one or more byproducts released by the T2Rs and SCCs as a result of their stimulation.

In other embodiments, the test medium contains both one or more agonists for T2Rs and/or SCCs of a human subject and one or more reagents which interact with one or more of the byproducts released by T2Rs and/or SCCs in a human subject as a result of stimulation. Preferably, a test medium contains one agonist and one reagent.

In a particularly preferred embodiment of the invention, the test kit contains one or more paper strips, which paper strips contain different agonists for T2Rs and/or SCCs of a human subject, and, when more than one paper strip of the test kit contain the same agonist and/or reagent in the same amount(s), those paper strips are in the same container, and paper strips with different agonist and/or reagents or different amounts thereof, are in different containers. In preferred test kits, there are one or more test strips of each type; there can be different numbers of test strips of different types. The number of types of test strips and the number of each type of test strip can vary depending on the treatment program or protocol (what is being monitored, length of treatment, and frequency of testing). Optionally, a test kit may have a unique identifier.

The methods and test kits of this invention can more accurately indicate an individual's susceptibility and clinical course to microbial infections and inflammatory disease for microbial infections and inflammatory diseases that correlate with T2R and/or SCC stimulation.

The genes encoding the TAS2Rs localize primarily to chromosomes 7 and 12, each with functional and non-functional polymorphisms. The correlation between taste receptor genetics and function and its potential role in sinusitis presentation and outcome was first characterized for the bitter taste receptor T2R38. The gene for this bitter receptor, TAS2R38, has two prevalent allele polymorphisms that correlate with taste receptor function. Functional alleles are characterized by a position 49 proline, position 262 alanine, and position 296 valine (known as a PAV genotype) versus alanine, valine, and isoleucine at the respective positions (AVI genotype) for the non-functional allele. Individuals who possess two functional alleles are considered as super tasters who are able to perceive bitter compounds such as propylthiouracil. Those with two AVI alleles are considered non-tasters and those with a single allele have heterogeneous taste perception.

Genetic and/or environmental modifiers could contribute, albeit to different degrees, to the definition of the phenotype throughout life. Regarding bitter taste receptors (T2Rs), changes in gene expression in the development phase or hormonal influences around the time of puberty may account for a different penetrance of the T2R38 gene, TAS2R38, genotypes at different ages. Taste receptor function varies from individual to individual due to genetic polymorphisms. While only a few of these polymorphisms have well-documented phenotypic effects, hundreds of T2R polymorphisms and several T1R polymorphisms have been noted in humans. The most well-known and well characterized example is the bitter receptor isoform T2R38. The TAS2R38 gene encoding T2R38 has two common polymorphisms, one encoding a functional receptor and one encoding a nonfunctional receptor. The differences in the resulting proteins are at amino acid positions 49, 262, and 296. The functional T2R38 receptor contains proline (P), alanine (A), and valine (V) residues while the nonfunctional T2R38 contains alanine (A), valine (V), and isoleucine (I) at these positions, respectively. Loss of the valine at the third position in the AVI variant prevents receptor activation.

These polymorphisms are distributed in a nearly Mendelian ratio in Caucasian populations. Homozygous AVI/AVI individuals (approximately 30% frequency in Caucasian populations) are “non-tasters” for the T2R38-specific agonists' phenylthiocarbamide (also known as phenylthiourea) and propylthiouracil. PAV/PAV individuals (approximately 20% frequency in Caucasian populations) are termed “super tasters” for these agonists because they perceive them as intensely bitter, while AVI/PAV heterozygotes have varying intermediate levels of taste. In some individuals, the T2R38 receptor contains alanine (A), alanine (A), and valine (V). Furthermore, individual differences in the expression of the PAV (Proline, Alanine, Valine) haplotype among heterozygous may account for the variation in bitter taste perception. Consequently, a continuum of intermediate levels of responsiveness probably separate the insensitive phenotype from the hypersensitive phenotype.

Genetic variations in taste receptor functionality cause differential responsiveness in cells isolated from different individuals, and corresponding taste receptor function correlates with disease severity in chronic rhinosinusitis (CRS). This has been best characterized for patients who are homozygous for the non-functional variant of T2R38. They are more likely to require surgical intervention for CRS, and more likely to develop a Gram-negative infection. Recent work has shown that phenotypic taste tests with denatonium, a broad T2R agonist, and sucrose, a T1R2/3 agonist, can reflect clinical disease status in CRS and partially stratify control subjects and CRS patients. It is thought that patients with CRS possess hypo-responsive bitter taste receptors, rating denatonium as less bitter than controls, while also possessing hypersensitive sweet taste receptors, which compounds the reduced antimicrobial response to sinonasal pathogens.

Bitter taste receptor phenotype appears to associate with clinical course following infection. Each individual's susceptibility to bacterial infections, viral infections, and inflammatory diseases may be predicted in correlation with the level of phenotypic expression of T2Rs and SCCs. While one is able to evaluate for receptor status via genetic analysis, the level of phenotypic expression appears to better predict the clinical course of infection, and prior studies have shown decreasing levels of phenotypic expression with increasing age. The multiple currently available taste tests to assess the level of phenotypic expression, which show subjective results, are often misinterpreted.

TAS2Rs line the tongue, but are also expressed on the surface of ciliated epithelial cells of the upper respiratory tract. TAS2Rs make up part of the innate immune system and the function of specific TAS2Rs is genetically determined with almost equal prevalence of functional and non-functional genotypes in the population. At the present time, it is thought that the repertoire of T2Rs expressed on ciliated cells and the repertoire of T2Rs expressed on SCCs are mutually exclusive. Recent work demonstrates that the NO-producing T2R response is exclusively found in ciliated cells, while production of antimicrobial peptides is driven only by T2Rs on SCCs.

In the airway, taste receptors are present on a variety of cell types and have been shown to mediate several complementary components of innate immune defense. For example, ciliated sinonasal epithelial cells express T2R38 and respond to PTC and acyl-homoserine lactones, bitter compounds released by gram-negative bacteria such as Pseudomonas aeruginosa. Activation of T2R38 triggers an increase in intracellular calcium (Ca²⁺) yielding stimulation of NO synthase with resultant production of intracellular NO. The NO, through cyclic GMP, increases ciliary beat frequency (CBF) and diffuses into the mucus layer where it has direct bactericidal activity.

One subset of T2Rs, when activated, stimulates the respiratory epithelium to generate NO, while a second subset of T2Rs expressed on solitary chemosensory cells (SCCs), can stimulate release of antimicrobial peptides. Both T2R-mediated pathways are considered integral to the upper airway innate immune defense system.

The TAS2R38 allelic makeup directly correlates with ability to generate NO in response to TAS2R38 stimulation and clear P. aeruginosa from explanted sinonasal ciliated cells.

Stimulation of ciliated nasal epithelial cells with agonists induces release of nitric oxide (NO). Release of NO from epithelial cells has been shown to reduce the growth of pathogenic bacteria.

With regard to whether NO may be helpful in the treatment of SARS-CoV-2 infection, SARS-CoV replication is inhibited by NO when supplied by the NO donor molecule S-nitroso-N-acetylpenicillamine. NO also prevented maturation of the viral S protein of SARS-CoV through inhibition of palmitoylation that results in reduced binding and viral fusion, leading to reduced capacity for viral entry.

NO is also able to inhibit viral replication of numerous viruses including the severe acute respiratory syndrome coronavirus (SARS-CoV and SARS-CoV-2). As the nasal airway is a primary portal of entry for the SARS-CoV-2 virus, it is postulated that SARS-CoV-2 infection via the nasal route may be suppressed by agonist-induced NO production from ciliated nasal epithelial cells, akin to NO suppression of SARS-CoV, leading to decreased incidence and severity of SARS-CoV-2 infection.

Improvement of the innate immune response to prevent infection within the nasal cavity has potential to reduce the burden of chronic rhinosinusitis (CRS), along with other viral upper respiratory inoculations. It has been shown that stimulation of nasal epithelial cells with phenylthiocarbamate (PTC), another T2R agonist, induces transmembrane calcium fluxes that correlate with NO release. This stimulation is associated with reductions in bacterial growth when nasal epithelial cells are overlaid with Pseudomonas aeruginosa. Exposure to NO has been shown to inhibit replication for many DNA and RNA viruses including hantavirus and the murine hepatitis coronavirus.

Similarly, SCCs, rare epithelial cells that express both T1R2/3 and T2R receptors, also respond to bitter compounds secreted by bacteria in the upper airway. Stimulation of T2Rs on the surface of human SCCs by the bitter agonist denatonium elicits a calcium response that spreads via gap junctions to neighboring epithelial cells, triggering a release of pre-formed stores of antimicrobial peptides.

In some embodiments, the methods of the invention are repeated one or more times, and can be used to obtain a data set. The methods and test kits of the invention can be used at regular time intervals, as listed above, to evaluate changing and/or current levels of phenotypic expression of T2Rs and SCCs. Because it is known that the level of phenotypic expression of T2Rs and SCCs decreases with age, this the methods of this invention can help predict susceptibility and clinical course over time as a patient ages, or for a patient over time for other purposes.

Another application is to evaluate current levels of phenotypic expression, which may help predict susceptibility to, and clinical course of, seasonally-occurring infections.

Agonist stimulation of T2Rs will result in increased levels of phenotypic expression, which is measurable via one or more of the methods of this invention; preferably, taste response of the human subject is used as the indicator of phenotype. Stimulation of the T2Rs and/or SCCs can be acute for activation, longer for increased function. Longer-term stimulation of T2Rs and/or SCCs can increase the level of expression and functionality of T2Rs and/or SCCs for many tasters, but not usually for super-tasters. Non-tasters for T2R38 may have other T2Rs and/or SCCs stimulated with one or more agonists to increase their phenotypic expression of T2Rs and/or SCCs other than T2R38. Generally, changes in the level of phenotype expression (and taster level) vary with the agonist(s) used, duration of use, and initial phenotype expression.

Stimulation of T2Rs and/or SCCs can increase immune response, and one or more agonists can be applied to a human subject to achieve an increased immune response on an acute basis or for a longer term. An acute basis ranges from a one-time application to or more repeated applications of one or more agonists over about 12 days. Longer term is about 12 days or longer, and can last for years. These stimulations to increase immune response employ one or more therapeutic agonists.

The following examples are presented for purposes of illustration, and are not intended to impose limitations on the scope of this invention.

### Example 1

In a clinical study of 1935 human patients who submitted to a taste test for the T2Rs, an expected distribution of approximately 25% strong reaction patients, 50% intermediate reaction patients, and 25% no-reaction patients was found. The study followed the 1935 patients for three months to observe their outcomes with the COVID-19 virus (SARS-CoV-2). A significant correlation was found between bitter taste receptor reaction and COVID-19.

Patients exposed to SARS-CoV-2 were enrolled from an outpatient clinical practice and an inpatient hospital from July 2020 through August 2020 and were followed prospectively until Sep. 30, 2020. All patients underwent phenotype taste testing, and each patient's SARS-CoV-2 status was confirmed via polymerase chain reaction (PCR), immunoglobulin M (IgM) and immunoglobulin G (IgG) testing to confirm absence of infection. Patients underwent study inclusion with phenotype taste testing and evaluation of lack of infection with SARS-CoV-2 via PCR (to exclude current infection), IgM and IgG (to exclude previous infection). Patients with evidence of active infection with SARS-CoV-2 via PCR at study commencement were excluded. Also excluded were patients with evidence of prior infection with SARS-CoV-2 via IgM and/or IgG at study commencement.

Levels of phenotypic expression of a T2R, specifically TAS2R38 (also referred to as T2R38), was determined via commercially-available paper taste test strips to evaluate the genetically determined taste response phenotype of each subject. These tests included four separate taste test strips administered in the following order:

1. a control (no taste chemical),

2. phenylthiocarbamide (PTC, 3 to 5 μg),

3. thiourea, and

4. sodium benzoate.

The sodium benzoate taste test strip was used to help control for potential for global loss of taste associated with SARS-CoV-2. Patients with positive results to the control taste test strip were excluded from the study.

In the series of taste tests, the patients were each instructed to place the provided litmus paper taste test strip on their tongue until completely moistened, then the next litmus paper strip was provided in the order stated above. Sips of water were permitted between the application of each test strip. Patients were instructed to comment on the quality of taste they perceived and to comment on the intensity of the taste on a visual analog scale from 0 to 10, where 0 indicates no perception of taste and 10 indicates extremely intense taste quality perceived, as compared to the control taste test paper. Each patient was oriented to the scale with a verbal explanation prior to proceeding with the test.

All patients included in the study were categorized into 3 groups (supertasters, tasters, & non-tasters) via their level of phenotypic expression of T2R38. Scores of 0 and 1 to PTC were classified as non-tasters; scores of 2 to 8 to PTC were classified as tasters; and scores of 9 and 10 to PTC were classified as supertasters. Scores for thiourea were used to confirm the level of phenotypic expression of T2R38 primarily in tasters. A randomized subgroup of patients in the study underwent genotype analysis by Monell Chemical Senses Center, Philadelphia, Pa., USA, using Oragene® DNA collection kits and DNA Genotek® genetic testing kits to correlate phenotype.

Patients were followed until infection with SARS-CoV-2, as confirmed by PCR. Phenotype expression of T2R38 via the taste testing protocol described above was re-determined after infection and the results of both genotype and phenotype were correlated with clinical course and outcome of disease. Patients were stratified into more severe and less severe clinical course of disease according to need for hospitalization during their infected period. Patients requiring hospitalization for treatment comprise the more severe cohort. Outcomes related to severity of disease (days symptomatic and need for hospitalization) were assessed via medical records.

Statistical analyses were performed using SAS analytical software version 9.4. Unadjusted comparisons of baseline characteristics and outcomes were conducted via chi-square tests and analyses of variance. Logistic regression analyses and zero-inflated Poisson analysis were used to assess relationships between tasting phenotypes and outcomes; all models were adjusted for age and sex. All aspects of this study were reviewed and approved by the Baton Rouge General Institutional Review Board (IRB00005439).

Overall, 1935 subjects (mean age, 45.5 years; 56.9% female) participated in the study, with 510 (26.4%) being non-tasters, 917 (47.4%) tasters, and 508 (26.3%) supertasters, as determined by a subjective taste test (see Table 1). Results of the taste test suggest a decreasing level of phenotypic expression with increasing age (p<0.0001) among supertasters (mean age, 41.6 years), tasters (mean age, 45.6 years), and non-tasters (mean age, 49.1 years). Table 1 summarizes some of the baseline characteristics and outcomes of patients exposed to SARS-CoV-2.

TABLE 1

| Patients | Overall | | Non-Tasters | | Tasters | | Supertasters | | p-value |
|---|---|---|---|---|---|---|---|---|---|
| N (%) | 1935 | (100) | 510 | (26.4) | 917 | (47.4) | 508 | (26.3) | |
| Baseline characteristics | | | | | | | | | |
| Age, years, mean (SD) | 45.5 | (13.9) | 49.1 | (15.9) | 45.6 | (13.4) | 41.6 | (11.2) | <0.0001 |
| Sex, n (%) | | | | | | | | | <0.0001 |
| Female | 1101 | (56.9) | 290 | (56.9) | 467 | (50.9) | 344 | (67.7) | |
| Male | 834 | (43.1) | 220 | (43.1) | 450 | (49.1) | 164 | (32.3) | |
| Outcomes | | | | | | | | | |
| SARS-CoV-2 positive, n (%) | 266 | (13.8) | 147 | (55.3) | 104 | (39.1) | 15 | (5.6) | <0.0001 |
| Hospitalization*, n (%) | 55 | (20.7) | 47 | (85.5) | 8 | (14.6) | 0 | (0) | <0.0001 |
| Symptom duration*, days, | 18.7 | (7.7) | 23.7 | (5.2) | 13.5 | (4.8) | 5.0 | (2.0) | <0.0001 |
| mean (SD) | | | | | | | | | |

*Among patients positive for SARS-CoV-2.

During the study period, 266 (13.8%) patients tested positive for SARS-CoV-2 via PCR. Of these, 55 (20.7%) patients required hospitalization. Symptom duration among positive cases ranged from 0 to 48 days. Non-tasters were significantly more likely to test positive for SARS-CoV-2 (p<0.0001), to be hospitalized once infected (p=0.0055), and to be symptomatic for a longer duration (p<0.0001; see Table 2). Risk of infection and of symptom duration showed significant evidence of linear trends across the tasting phenotypes. Table 2 shows the relationships between taster classification, SARS-CoV-2 infection, and clinical consequences, where SE stands for standard error and CI stands for confidence interval.

TABLE 2

| | | | | | | | | Symptom | |
|---|---|---|---|---|---|---|---|---|---|
| | % positive for SARS-CoV-2 | | | | % Hospitalized | | | duration, days | |
| Patients | Mean (SE)¹ | | OR (95% CI) | | Mean (SE) ¹ | | OR | Mean (SE) ¹ | |
| Non-taster | 25.8 | (2.0) | 10.1 | (5.8-17.8) | 12.9 | (3.5) | 3.9 (1.5-10.2) | 21.2 | (0.5) |
| Taster | 10.5 | (1.0) | 3.4 | (1.9-6.0) | 3.7 | (1.8)³ | ref³ | 13.2 | (0.4) |

| | | | | | |
|---|---|---|---|---|---|
| Supertaster | 3.3 | (0.8) | ref | 5.5 | (0.6) |

| | | | |
|---|---|---|---|
| p-value | <0.0001² | 0.0055 | <0.0001² |

¹Least-square mean estimates from models adjusting for age and sex.
²Test for linear trend (dose-response relationship) across taster phenotypes.
³Tasters and supertasters were pooled for analysis because there were no hospitalizations among supertasters.

### Example 2

An investigational device study was performed at an outpatient clinical practice and inpatient hospital on 171 patients and health care workers. All subjects were categorized into 3 groups (high tasters, moderate tasters, and low/non-tasters) via their level of phenotypic expression of T2Rs, tested using taste strip tests as described in Example 1. Subjects underwent genotype analysis to detect single nucleotide polymorphism (SNP) in the TAS2R38 gene. Three polymorphisms were genotyped using real-time PCR single nucleotide polymorphism genotyping assays (rs713598, rs1726866, and rs10246939). Correlation between level of phenotypic expression and genotype was conducted.

In our study, we categorized any subject with two copies of the PAV allele as high taster, those with one copy of PAV allele as moderate taster, and finally, those with no PAV alleles in their genotype were classified as low/nontasters.

Participants with evidence of active infection with SARS-CoV-2 via PCR at study commencement were excluded. Participants with evidence of prior infection with SARS-CoV-2 via IgM and or IgG at study commencement were excluded. Participants were excluded from evaluation with positive results to the Control strip.

Statistical analyses were performed using SPSS v 22 (SPSS Statistics for Windows, version 22.0; IBM, Armonk, N.Y.). Descriptive data are presented as percentages and means±standard deviation (SD). Kendall's tau-B was used for ordinal values. Chi-squared analysis was used for relationships of nominal variables. Student t test (2-tailed) was used for comparisons of parametric data. Results were deemed significant with a p value of <0.05.

One hundred seventy-one patients (171; 53.2% female) with a mean age of 41.56 years; were assessed with phenotype taste testing. All participants were categorized into 3 groups (high tasters, moderate tasters, and low/nontasters) via their level of phenotypic expression of T2R. Thirty-six (21.1%) were categorized as high tasters. Ninety-one (53.2%) were categorized as moderate tasters. Forty-four (25.7%) were categorized as low/nontasters (Table 3).

Genetic analysis of the 171 subjects revealed the PAV/PAV diplotype in 39 subjects, while 90 subjects were classified as heterozygotes (PAV/AVI & PAV/AAV) (79 PAV/AVI and 11 PAV/AAV). 42 subjects were categorized in the non-PAV containing group (37 AVI/AVI and 5 AVI/AAV), where AAV is alanine-alanine-valine.

When evaluating the relationship between phenotype and genotype, phenotype showed 94.7% (162/171) accuracy in predicting genotype (p-value<0.01). The average age of the discordant subjects was 61.3. Rate of discordant results in high taster group was lower than the other two groups, with 35/36 (97.2%), with only 1 case testing phenotypically as a high taster but displayed a PAV/AVI genotype. Our study showed discordant results in the moderate taster group (single PAV allele) higher than the other 2 groups, where 86/91 single-PAV carrying subjects (75 PAV/AVI and 11 PAV/AAV) tested phenotypically as moderate tasters (94.5%), with disagreement in 5 cases (4 PAV/PAV and 1 AVI/AAV). Lastly in the low/nontaster group, 41/44 subjects (37 AVI/AVI and 4 AVI/AAV) tested phenotypically in the low/nontaster group (93.2%), with 3 cases that displayed the PAV allele and still categorized phenotypically as a low/nontaster.

In the nine subjects with discordant results, seven subjects (5 males and 2 females, average age 68.6 years) had a genotype of a higher group (PAV/PAV) but tested phenotypically in the lower group (moderate taster); this is set forth in Table 4.

TABLE 3

| | Phenotype | | | | | 95% |
|---|---|---|---|---|---|---|
| | High | Moderate | | | % | Confidence |
| Genotype | taster | Taster | Low/NonTaster | Total | Accuracy | Interval |
| PAV/PAV | 35 | 4 | | 39 | 35/36 | ±0.044 |
| | | | | | (97.2%) | |
| PAV/AVI | 1 | 75 | 3 | 79 | 86/91 | ±0.054 |
| PAV/AAV | | 11 | | 11 | (94.5%) | |
| AVI/AVI | | | 37 | 37 | 41/44 | ±0.047 |
| AVI/AAV | | 1 | 4 | 5 | (93.2%) | |
| Total | 36 | 91 | 44 | 171 | 162/171 | |
| | | | | | (94.7%) | |

TABLE 4

| Number | Age | Sex | Phenotype | Genotype |
|---|---|---|---|---|
| 1 | 46 | Female | High taster | PAV/AVI |
| 2 | 26 | Female | Moderate Taster | AVI/AAV |
| 3* | 81 | Male | Moderate Taster | PAV/PAV |
| 4* | 78 | Female | Moderate Taster | PAV/PAV |
| 5* | 51 | Female | Moderate Taster | PAV/PAV |
| 6* | 62 | Male | Moderate Taster | PAV/PAV |
| 7* | 65 | Male | Low/NonTaster | PAV/AVI |
| 8* | 70 | Male | Low/NonTaster | PAV/AVI |
| 9* | 73 | Male | Low/NonTaster | PAV/AVI |

*A subject with a lower level of phenotypic expression of T2Rs in comparison to their genotype (seven subjects in total).

Further embodiments of this invention include, without limitation:

A) A test method comprising
stimulating T2Rs and/or SCCs of a human subject with one or more agonists, and
detecting one or more byproducts released by the T2Rs and/or SCCs as a result of stimulation of the T2Rs and/or SCCs, wherein the detecting of the byproducts comprises a method comprising either
   I-i) applying one or more reagents which interact with one or more of the byproducts released by the stimulation of the T2Rs and/or SCCs, and
   I-ii) detecting the interaction of the reagent with one or more byproducts, optionally by employing chemiluminescence, an electrochemical sensor, or an optical sensor, to detect the interaction of one or more reagents with one or more of the byproducts, or
   II) employing chemiluminescence, an electrochemical sensor, or an optical sensor to detect one or more of the byproducts released by the stimulation of the T2Rs and/or SCCs.

B) The test method as in A) wherein the agonists are separately applied prior to the reagent which interacts with one or more of the byproducts released by T2Rs and/or SCCs as a result of stimulation of T2Rs and/or SCCs.

C) The test method as in A) wherein the agonist and the reagent that interacts with one or more byproducts released by T2Rs and/or SCCs as a result of stimulation of T2Rs and/or SCCs are contained on or the same test medium.

D) The test method as in A) wherein the agonists are selected from the group consisting of caffeine, denatonium, strychnine, quinine, terpenes, phenylthiocarbamate, thiourea, sodium benzoate, and any two or more of the foregoing.

E) The test method as in A) wherein the detecting of the byproducts comprises applying a test medium containing the reagents which interact with the byproducts.

F) The test method as in E) wherein the reagents which interact with the byproducts released by T2Rs and/or SCCs as a result of stimulation of T2Rs and/or SCCs comprise a Griess reagent.

G) The test method as in A) wherein the agonist(s) are selected from caffeine, denatonium, strychnine, quinine, terpenes, phenylthiocarbamate, thiourea, sodium benzoate, and any two or more of the foregoing, and wherein the detecting of the byproducts comprises applying a test medium containing the reagents which interact with the byproducts.

H) The test method as in G) wherein the reagents which interact with the byproducts released by T2Rs and/or SCCs as a result of stimulation of T2Rs and/or SCCs comprise a Griess reagent.

I) A test method for determining a level of phenotypic expression of T2Rs and/or SCCs in a human subject, the method comprising
i) stimulating T2Rs and/or SCCs of a human subject by exposing at least a portion of oral or nasal tissue of the human subject to one or more agonists,
ii) recording a discerned level of taste perception by the human subject after the stimulation, and
iii) correlating the discerned level of taste perception to the level of phenotypic expression of the T2Rs and/or SCCs of the human subject.

J) The method according to I) further comprising repeating steps i)-iii) one or more times, and wherein the agonists comprise a therapeutic agonist.

K) The method according to I) further comprising
repeating steps i)-iii) one or more times to obtain a data set,
and optionally performing trend analysis on the data set,
wherein the agonists comprise a therapeutic agonist.

L) The method according to J) wherein the repeating step is performed two or more times, at regular time intervals.

M) The method according to L) wherein the time intervals each are 8 hours, daily, weekly, biweekly, monthly, bimonthly, semiannually, annually, or biannually.

N) The method according to I) wherein the agonists are selected from the group consisting of caffeine, denatonium, strychnine, quinine, terpenes, phenylthiocarbamate, thiourea, sodium benzoate, and any two or more of the foregoing.

O) The method according to J) wherein the therapeutic agonist is selected from the group consisting of caffeine, denatonium, strychnine, quinine, xylitol, grapefruit seed extract or naringenin, a terpene, and any two or more of the foregoing.

P) A method according to I) wherein:
steps i) and ii) are repeated one or more times, and the stimulating by each of one or more different agonists is sequential,
the recording of each discerned level of taste perception by the human subject occurs after each stimulation, and
the correlating is of one or more of the discerned levels of taste perception to the level of phenotypic expression of the T2Rs and/or SCCs of the human subject.

Q) The method according to P) wherein the correlating comprises employing a computer processor programmed with machine-readable instructions causing the computer processor to:
a) receive and store the discerned levels of taste perception with respect to each agonist,
b) ascribe a weighting to each of the agonists according to their known stimulation of T2Rs and/or SCCs,
c) calculate a weighted taste perception from the discerned level of taste perception by multiplying the ascribed weighting and discerned level of taste perception for each agonist applied, to produce an aggregated, weighted level of taste perception which indicates the level of phenotypic expression.

R) The method according to P) wherein the agonists are selected from the group consisting of caffeine, denatonium, strychnine, quinine, terpenes, phenylthiocarbamate, thiourea, sodium benzoate, and any two or more of the foregoing.

S) A method for evaluating a level of expression or functionality of T2Rs and/or SCCs in a human subject, the method comprising
stimulating the T2Rs and/or SCCs of the human subject with one or more agonists, and
detecting one or more byproducts, if any, released by the T2Rs and/or SCCs as a result of stimulation of the T2Rs and/or SCCs by exposing the breath, saliva or respiratory mucosa of the human subject to the detecting, wherein the detecting of the byproducts comprises a method comprising either
   I-i) applying one or more reagents which interact with one or more of the byproducts released by the stimulation of the T2Rs and/or SCCs, and
   I-ii) detecting the interaction of the reagent with one or more byproducts, optionally by employing chemiluminescence, an electrochemical sensor, or an optical sensor, to detect the interaction of one or more reagents with one or more of the byproducts, or
   II) employing chemiluminescence, an electrochemical sensor, or an optical sensor to detect one or more of the byproducts released by the stimulation of the T2Rs and/or SCCs,
and optionally discerning a level of byproducts produced by the stimulation of the T2Rs and/or SCCs, indicative of level of expression or functionality of the T2Rs and/or SCCs, which level of expression or functionality of the T2Rs and/or SCCs is correlated to the level of phenotypic expression.

T) The method according to S) wherein the agonists are selected from the group consisting of caffeine, denatonium, strychnine, quinine, terpenes, phenylthiocarbamate, thiourea, sodium benzoate, and any two or more of the foregoing.

U) The method according to S) wherein the detecting of the byproducts comprises applying a test medium containing one or more reagents which interact with one or more of the byproducts.

V) The method according to U) wherein the reagent which interacts with one or more byproducts released by T2Rs and/or SCCs as a result of stimulation of T2Rs and/or SCCs comprise a Griess reagent.

W) The method according to S) wherein the agonist(s) are selected from caffeine, denatonium, strychnine, quinine, terpenes, phenylthiocarbamate, thiourea, sodium benzoate, and any two or more of the foregoing, and wherein the detecting of the byproducts comprises applying a test medium containing one or more reagents which interact with one or more of the byproducts.

X) The method according to W) wherein one or more of the reagents which interact with one or more byproducts released by T2Rs and/or SCCs as a result of stimulation of T2Rs and/or SCCs comprise a Griess reagent.

Y) A test kit comprising at least one test medium containing (a) one or more agonists for T2Rs and/or SCCs of a human subject and/or (b) one or more reagents which interact with one or more byproducts released by T2Rs and/or SCCs of a human subject as a result of stimulation of the T2Rs and/or SCCs.

Z) The test kit according to Y) wherein the agonists are selected from the group consisting of caffeine, denatonium, strychnine, quinine, terpenes, phenylthiocarbamate, thiourea, sodium benzoate, and any two or more of the foregoing.

Components referred to by chemical name or formula anywhere in the specification or claims hereof, whether referred to in the singular or plural, are identified as they exist prior to coming into contact with another substance referred to by chemical name or chemical type (e.g., another component, a solvent, or etc.). It matters not what chemical changes, transformations and/or reactions, if any, take place in the resulting mixture or solution as such changes, transformations, and/or reactions are the natural result of bringing the specified components together under the conditions called for pursuant to this disclosure. Thus the components are identified as ingredients to be brought together in connection with performing a desired operation or in forming a desired composition. Also, even though the claims hereinafter may refer to substances, components and/or ingredients in the present tense (“comprises”, “is”, etc.), the reference is to the substance, component or ingredient as it existed at the time just before it was first contacted, blended or mixed with one or more other substances, components and/or ingredients in accordance with the present disclosure. The fact that a substance, component or ingredient may have lost its original identity through a chemical reaction or transformation during the course of contacting, blending or mixing operations, if conducted in accordance with this disclosure and with ordinary skill of a chemist, is thus of no practical concern.

The invention may comprise, consist, or consist essentially of the materials and/or procedures recited herein.

As used herein, the term “about” modifying the quantity of an ingredient in the compositions of the invention or employed in the methods of the invention refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term about also encompasses amounts that differ due to different equilibrium conditions for a composition resulting from a particular initial mixture. Whether or not modified by the term “about”, the claims include equivalents to the quantities.

Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

This invention is susceptible to considerable variation in its practice. Therefore the foregoing description is not intended to limit, and should not be construed as limiting, the invention to the particular exemplifications presented hereinabove.

1. (canceled)

2. (canceled)

3. (canceled)

4. (canceled)

5. (canceled)

6. (canceled)

7. (canceled)

8. (canceled)

9. A test method for determining a level of phenotypic expression of T2Rs and/or SCCs in a human subject, the method comprising
i) stimulating T2Rs and/or SCCs of a human subject by exposing at least a portion of oral or nasal tissue of the human subject to one or more agonists,
ii) recording a discerned level of taste perception by the human subject after the stimulation, and
iii) correlating the discerned level of taste perception to the level of phenotypic expression of the T2Rs and/or SCCs of the human subject.

10. The method according to claim 9 further comprising repeating steps i)-iii) one or more times, and wherein the agonists comprise a therapeutic agonist.

11. The method according to claim 9 further comprising repeating steps i)-iii) one or more times to obtain a data set, and optionally performing trend analysis on the data set, wherein the agonists comprise a therapeutic agonist.

12. The method according to claim 10 wherein the repeating step is performed two or more times, at regular time intervals.

13. The method according to claim 12 wherein the time intervals each are 8 hours, daily, weekly, biweekly, monthly, bimonthly, semiannually, annually, or biannually.

14. The method according to claim 9 wherein the agonists are selected from the group consisting of caffeine, denatonium, strychnine, quinine, terpenes, phenylthiocarbamate, thiourea, sodium benzoate, and any two or more of the foregoing.

15. The method according to claim 10 wherein the therapeutic agonist is selected from the group consisting of caffeine, denatonium, strychnine, quinine, xylitol, grapefruit seed extract or naringenin, a terpene, and any two or more of the foregoing.

16. A method according to claim 9 wherein:
steps i) and ii) are repeated one or more times, and the stimulating by each of one or more different agonists is sequential,
the recording of each discerned level of taste perception by the human subject occurs after each stimulation, and
the correlating is of one or more of the discerned levels of taste perception to the level of phenotypic expression of the T2Rs and/or SCCs of the human subject.

17. The method according to claim 16 wherein the correlating comprises employing a computer processor programmed with machine-readable instructions causing the computer processor to:
a) receive and store the discerned levels of taste perception with respect to each agonist,
b) ascribe a weighting to each of the agonists according to their known stimulation of T2Rs and/or SCCs,
c) calculate a weighted taste perception from the discerned level of taste perception by multiplying the ascribed weighting and discerned level of taste perception for each agonist applied, to produce an aggregated, weighted level of taste perception which indicates the level of phenotypic expression.

18. The method according to claim 16 wherein the agonists are selected from the group consisting of caffeine, denatonium, strychnine, quinine, terpenes, phenylthiocarbamate, thiourea, sodium benzoate, and any two or more of the foregoing.

19. (canceled)

20. (canceled)

21. (canceled)

22. (canceled)

23. (canceled)

24. (canceled)

25. (canceled)

26. (canceled)
